(19) United States
(12) Patent Application Publication
(10) Pub. No.: US 2024/0393203 A1
(43) Pub. Date: Nov. 28, 2024

(54) PHYSIOLOGICAL INFORMATION MEASUREMENT DEVICE, PHYSIOLOGICAL INFORMATION MEASUREMENT METHOD, AND NON-TRANSITORY COMPUTER READABLE STORAGE MEDIUM

(71) Applicant: NIHON KOHDEN CORPORATION, Tokyo (JP)

(72) Inventors: Mariko Ito, Tokorozawa-shi (JP); Hirohito Igarashi, Tokorozawa-shi (JP); Fumiyuki Matsumura, Tokorozawa-shi (JP); Minori Hosoi, Tokorozawa-shi (JP)

(73) Assignee: NIHON KOHDEN CORPORATION, Tokyo (JP)

(21) Appl. No.: 18/671,340

(22) Filed: May 22, 2024

(30) Foreign Application Priority Data
May 25, 2023 (JP) 2023-085932

(51) Int. Cl. G01M 3/26; A61B 5/00

(52) U.S. Cl. CPC G01M 3/26; A61B 5/6892; A61B 2562/0247; A61B 2562/04

(57) ABSTRACT

A physiological information measurement device includes a sensor connected to an airbag containing air, the sensor being configured to measure pressure received by the airbag from a subject, and a processor configured to process a measurement result of the sensor to obtain information on a dynamic pressure and a static pressure of the subject, and a detector configured to detect an abnormality in a path from the airbag to the sensor, based on a change in the dynamic pressure and/or the static pressure.

### CROSS-REFERENCE TO RELATED APPLICATIONS

This application is based on and claims priority under 35 USC 119 from Japanese Patent Application No. 2023-085932 filed on May 25, 2023, the entire content of which is incorporated herein by reference.

### TECHNICAL FIELD

The presently disclosed subject matter relates to a physiological information measurement device, a physiological information measurement method, and a non-transitory computer readable storage medium.

### BACKGROUND ART

In a medical facility such as a hospital, it may be necessary to noninvasively measure physiological information such as a heartbeat and respiration of a patient lying on a bed without moving the patient. In a nursing home or the like, it is necessary to prevent a user from walking around and ensure safety of the user by a limited number of staff. To cope with such situations, there is known a technique (for example, JP2004-159804A) in the related art, in which an airbag is spread under a mattress laid on a bed, pressure applied to the airbag from a lying patient is detected by a pressure sensor, a microphone, or the like, and a heartbeat, respiration, or the like of the patient on the bed is measured. In this technique, whether the patient gets in or out of bed is determined by comparing an amplitude of physiological information such as the detected respiration or heartbeat with a predetermined threshold. In addition, a product has been developed in recent years, which analyzes states of a patient on a bed such as sleeping and awake, getting in bed and out of bed, in bed and out of bed, and changes in respiration and heartbeat from a measured body motion waveform, and notifies a medical worker of changes in conditions of the patient.

However, in the technique described in JP2004-159804A, when an abnormality (for example, leakage, tube detachment, occlusion) occurs in a measurement path (path from a bag of a physiological sensor 3 to a microphone 5), the abnormality of the measurement path may fail to be detected since it is not possible to distinguish getting out of bed of the patient from the abnormality of the measurement path.

### SUMMARY OF INVENTION

Aspect of non-limiting embodiments of the present disclosure relates to provide a physiological information measurement device, a physiological information measurement method, and a non-transitory computer readable storage medium storing a physiological information measurement program that can detect an abnormality in a measurement path from an airbag to a pressure sensor.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a physiological information measurement device including:
a sensor connected to an airbag containing air, the sensor being configured to measure pressure received by the airbag from a subject;
a processor configured to process a measurement result of the sensor to obtain information on a dynamic pressure and a static pressure of the subject; and
a detector configured to detect an abnormality in a path from the airbag to the sensor, based on a change in the dynamic pressure and/or the static pressure.

According to an aspect of the present disclosure, there is provided a physiological information measurement device including:
a first body; and
a second body,
in which the first body includes:
   a sensor connected to an airbag containing air, and the sensor being configured to measure pressure received by the airbag from a subject;
   a processor configured to process a measurement result of the sensor to obtain information on a dynamic pressure and a static pressure of the subject; and
   a transmitter configured to transmit, to the second body, the information on the dynamic pressure and the static pressure of the subject, and
the second body includes:
   a receiver configured to receive, from the first body, the information on the dynamic pressure and the static pressure of the subject; and
   a detector configured to detect an abnormality in a path from the airbag to the sensor, based on a change in the dynamic pressure and/or the static pressure.

According to an aspect of the present disclosure, there is provided a physiological information measurement device for measuring physiological information on a subject, based on pressure received by an airbag containing air from the subject, the physiological information measurement device including:
an obtainer configured to obtain information on a dynamic pressure and a static pressure of the subject; and
a detector configured to detect an abnormality in a path from the airbag to a sensor, based on a change in the dynamic pressure and/or the static pressure, the sensor being connected to the airbag and configured to measure the pressure received by the airbag from the subject.

According to an aspect of the present disclosure, there is provided a physiological information measurement method including:
measuring, by a sensor, pressure received by an airbag containing air from a subject;
processing a measurement result in the measuring and obtaining information on a dynamic pressure and a static pressure of the subject; and
detecting an abnormality in a path from the airbag to the sensor, based on a change in the dynamic pressure and/or the static pressure.

### BRIEF DESCRIPTION OF DRAWINGS

Exemplary embodiment(s) of the present invention will be described in detail based on the following figures, wherein:

FIG. 1 is a block diagram illustrating a schematic configuration of a physiological information analysis system according to an embodiment;

FIG. 2 is a block diagram illustrating a schematic configuration of a physiological information measurement device in FIG. 1;

FIG. 3 is a block diagram illustrating a schematic configuration of an analyzer in FIG. 2;

FIG. 4 is a schematic diagram illustrating a display screen of an operation display in FIG. 2;

FIG. 5 is a flowchart illustrating an outline of a processing procedure of a physiological information measurement method by a physiological information analysis device according to an embodiment;

FIG. 6A is a graph illustrating an example of a change over time of a pressure signal in a case where a static pressure change due to air leakage is fast;

FIG. 6B is a graph illustrating an example of a change over time of a static pressure signal in a case where the static pressure change due to air leakage is fast;

FIG. 6C is a graph illustrating an example of a change over time of a dynamic pressure signal in a case where the static pressure change due to air leakage is fast;

FIG. 7A is a graph illustrating an example of a change over time of the pressure signal in a case where the static pressure change due to air leakage is slow;

FIG. 7B is a graph illustrating an example of a change over time of the static pressure signal in a case where the static pressure change due to air leakage is slow;

FIG. 7C is a graph illustrating an example of a change over time of the dynamic pressure signal in a case where the static pressure change due to air leakage is slow;

FIG. 8 is a graph illustrating a waveform change in a case where a tube is occluded;

FIG. 9 is a subroutine flowchart illustrating processing details of step S103 in the flowchart in FIG. 5;

FIG. 10 is a schematic diagram illustrating data display on a display;

FIG. 11 is a block diagram illustrating a schematic configuration of the physiological information measurement device in FIG. 1 according to a first modification;

FIG. 12 is a block diagram illustrating a schematic configuration of the physiological information measurement device in FIG. 1 according to a second modification; and

FIG. 13 is a block diagram illustrating a schematic configuration of the physiological information measurement device in FIG. 1 according to a third modification.

### DESCRIPTION OF EMBODIMENTS

Hereinafter, a physiological information measurement device, a physiological information measurement method, and a non-transitory computer readable storage medium storing a physiological information measurement program according to an embodiment of the presently disclosed subject matter will be described in detail with reference to the drawings. In the drawings, the same members are denoted by the same reference numerals. Dimensional ratios in the drawings are exaggerated for convenience of the description, and may be different from actual ratios.

### Configuration of Physiological Information Measurement System

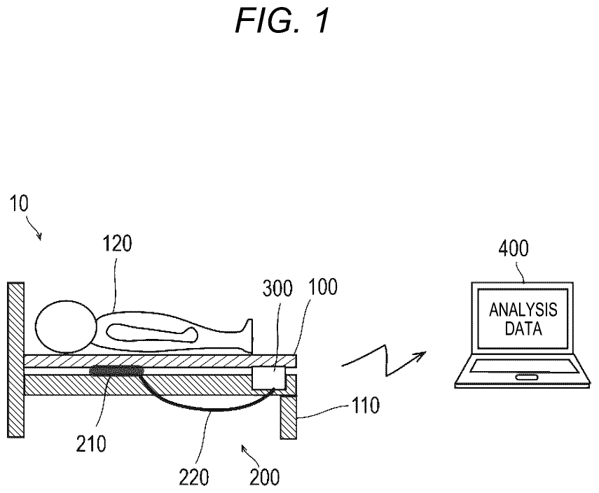

FIG. 1 is a block diagram illustrating a schematic configuration of a physiological information measurement system 10 according to an embodiment. In the present embodiment, it is assumed that in a medical facility such as a hospital, a nursing home, or the like, respiration and/or a heartbeat of a patient or a user (hereinafter, also referred to as a “subject”) 120 are measured in a state where the subject 120 is lying on a bed 110 on which a mattress 100 is laid. The physiological information measurement system 10 is a system mainly intended to noninvasively measure a respiratory rate of the subject 120 on the bed 110 and continuously display and record the measured respiratory rate, and may be used in, for example, a general hospital ward of a hospital.

The physiological information measurement system 10 can include a mat 210, a tube 220, and a physiological information measurement device 300.

The mat 210 can include a sealed airbag (not illustrated). The airbag can include an outlet for discharging air contained therein. One end of the tube 220 is coupled to the outlet. The air in the airbag is discharged to the physiological information measurement device 300 through the tube 220. The other end of the tube 220 is coupled to the physiological information measurement device 300. A length of the tube 220 is not limited, and the tube 220 may not come out of the physiological information measurement device 300. The airbag is formed of an airtight material (for example, resin and rubber) that is elastically deformable by pressure of the air contained therein. The airbag can include an elastic body including a compressible porous body (for example, sponge) therein, and air is taken in from outside by a restoring force of the elastic body to fill the airbag.

The mat 210 may be laid, for example, between a lower surface of the mattress 100 and an upper surface of a bed board of the bed 110, in a position corresponding to back (chest and abdomen) of the subject 120 on the bed 110 in a state in which the airbag is sufficiently filled with air. When the subject 120 lies on the bed 110 (gets in bed), load due to the subject 120 on the bed 110 and the mattress 100 and pressure due to a body motion of the subject 120 including a chest motion accompanying a respiratory motion and minute vibration accompanying a heartbeat motion are applied to the mat 210. Accordingly, a volume of the airbag changes according to the pressure received from the subject 120, so that a pressure change is transmitted to the physiological information measurement device 300 through the tube 220.

The mat 210 may be laid in a position corresponding to the back (chest and abdomen) of the subject 120 on the mattress 100 laid on the bed 110. In this case, the load due to the subject 120 on the bed 110 and the pressure due to the body motion of the subject 120 are applied to the mat 210.

### Configuration of Physiological Information Measurement Device 300

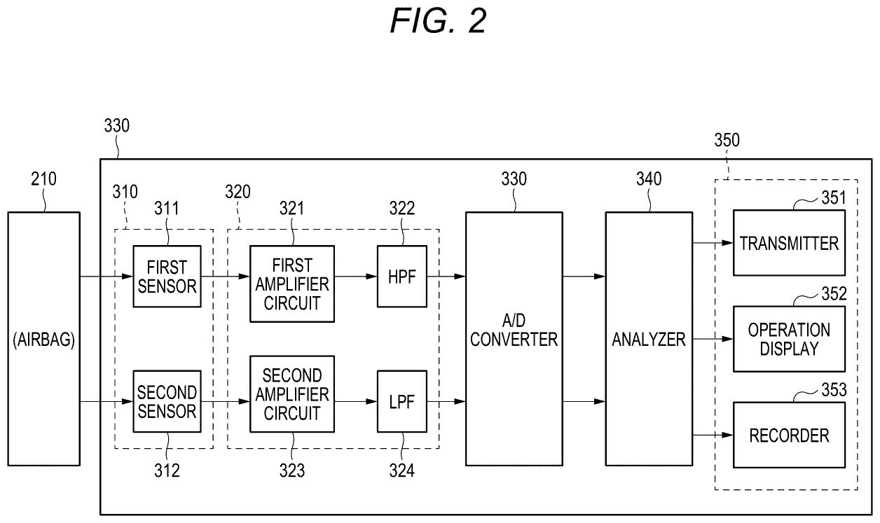

FIG. 2 is a block diagram illustrating a schematic configuration of the physiological information measurement device 300 in FIG. 1. The physiological information measurement device 300 can include a sensor 310, a signal processor 320, an A/D converter 330, an analyzer 340, and a system 350.

### Configuration of Sensor 310

The sensor 310 can include a first sensor 311 and a second sensor 312. The first sensor 311 is coupled to the other end of the tube 220, is configured to convert a pressure change of air from the airbag into an electric signal, and is configured to output the electric signal to the signal processor 320. The first sensor 311 is configured to measure a body motion component (hereinafter, referred to as “dynamic pressure”) including a respiratory motion and a heartbeat motion of the subject 120. The first sensor 311 may be a pressure sensor such as a gauge pressure sensor, a differential pressure sensor, an absolute pressure sensor, or a microphone sensor. In the present embodiment, a pressure sensor that is also configured to measure negative pressure is used, and a body motion change can be measured even when the negative pressure is generated.

The second sensor 312 is coupled to the other end of the tube 220, is configured to convert a pressure change of air from the airbag into an electric signal, and is configured to output the electric signal to the signal processor 320. The second sensor 312 may be a pressure sensor such as a gauge pressure sensor or an absolute pressure sensor. The second sensor 312 is configured to measure a change component of pressure (hereinafter referred to as “static pressure”) caused by a load change in the subject 120. Accordingly, when the first sensor 311 and the second sensor 312 are used at the same time, measurement accuracy of an AC component (that is, dynamic pressure) of the second sensor 312 may be low.

### Configuration of Signal Processor 320

The signal processor 320 is configured to process a measurement result of the sensor 310 to obtain information on the dynamic pressure and the static pressure of the subject 120. The signal processor 320 can include a first amplifier circuit 321, a high-pass filter (HPF) 322, a second amplifier circuit 323, and a low-pass filter (LPF) 324.

The first amplifier circuit 321 is configured to amplify the electric signal from the first sensor 311 to a predetermined voltage level and output the amplified signal. The HPF 322 is configured to extract an AC component having a predetermined first cutoff frequency fc1 or higher from the electric signal amplified by the first amplifier circuit 321, and output the AC component to the A/D converter 330. The first cutoff frequency fc1 is a frequency at which a frequency component corresponding to the dynamic pressure can pass while a frequency component corresponding to the static pressure being sufficiently cut off. For example, fc1 may be set to be a frequency at which a frequency component derived from respiration can pass. The frequency derived from respiration may be set to be, for example, about 0.1 to 0.4 [Hz] for an adult, based on information on the subject 120.

The second amplifier circuit 323 is configured to amplify the electric signal from the second sensor 312 to a predetermined voltage level and output the amplified signal. The LPF 324 is configured to extract an AC (or DC) component having a predetermined second cutoff frequency fc2 or lower from the electric signal amplified by the second amplifier circuit 323, and output the AC component to the A/D converter 330. The second cutoff frequency fc2 is a frequency at which a frequency component corresponding to the static pressure can pass while a frequency component corresponding to the dynamic pressure being sufficiently cut off. The first and second cutoff frequencies may be the same value or different values (fc1>fc2).

### Configuration of A/D Converter 330

The A/D converter 330 is configured to convert, into a digital signal of the dynamic pressure (information on the dynamic pressure), the electric signal (analog signal) of the dynamic pressure received from the HPF 322, and output the digital signal to the analyzer 340. The A/D converter 330 is configured to convert, into a digital signal of the static pressure (information on the static pressure), the electric signal (analog signal) of the static pressure received from the LPF 324, and output the digital signal to the analyzer 340.

### Configuration of Analyzer 340

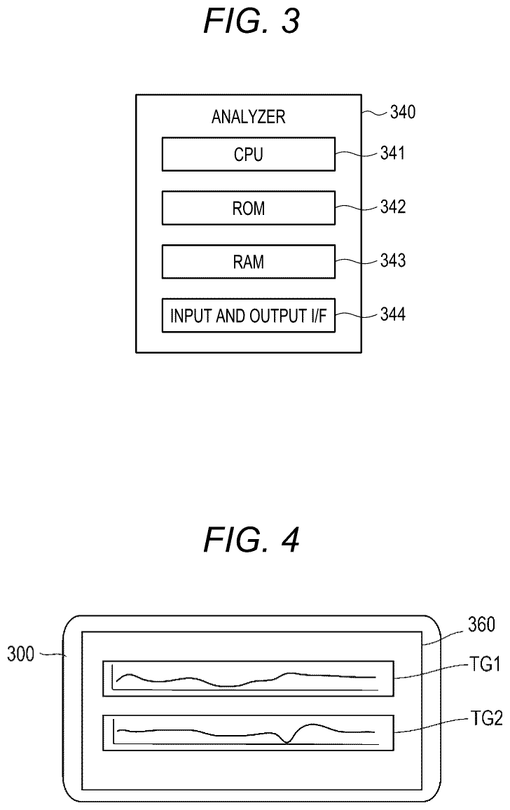

FIG. 3 is a block diagram illustrating a schematic configuration of the analyzer 340 in FIG. 2. The analyzer 340 can include a central processor (CPU) 341, a read only memory (ROM) 342, a random access memory (RAM) 343, and an input and output I/F 344. The CPU 341, the ROM 342, and the RAM 343 constitute a computer.

The CPU 341 implements various functions by loading a physiological information measurement program stored in advance in the ROM 342 into the RAM 343 and executing the program.

The ROM 342 is a non-volatile memory. The ROM 342 is configured to store programs such as an operating system (OS) and a physiological information measurement program, and various parameters necessary for calculation processing of the CPU 341. The ROM 342 can further include, for example, a solid state drive (SSD) or a hard disk drive (HDD) that stores a calculation result by the CPU 341 and a detection result of an abnormality in a measurement path.

The RAM 343 is a volatile memory and temporarily stores a determination result by the CPU 341 and various data.

The input and output I/F 344 is an input and output interface configured to transmit and receive data to and from the system 350.

The analyzer 340 is configured to calculate a respiratory waveform, a respiratory rate, a heartbeat waveform, and a heart rate of the subject 120, based on the digital signal of the dynamic pressure (hereinafter, referred to as a “dynamic pressure signal”). More specifically, the analyzer 340 is configured to generate a respiratory waveform based on a signal obtained by extracting a frequency component derived from respiration from the dynamic pressure signal, and calculate the respiratory rate of the subject 120 by a predetermined analysis algorithm for calculating the respiratory rate. For the extraction of the frequency component derived from respiration, for example, in a case where the subject 120 is an adult, a digital filter or the like that allows passage of a signal component of about 0.1 to 1.0 [Hz] can be used. In addition, the analyzer 340 is configured to generate a heartbeat waveform based on a signal obtained by extracting a frequency component derived from the heartbeat from the dynamic pressure signal, and calculate the heart rate of the subject 120 by a predetermined analysis algorithm for calculating the heart rate.

It is considered that magnitudes of the dynamic pressure signal and the static pressure signal differ depending on subjects. The analyzer 340 functions as a calculator, and is configured to calculate a standard value regarding each of the dynamic pressure signal and the static pressure signal of the subject 120. More specifically, the calculator is configured to calculate respective standard values, based on the dynamic pressure signal and the static pressure signal of the subject 120 obtained at a start of pressure measurement by the sensor 310 or at predetermined time intervals. Calculating the standard value can be used as a reference for determining validity of a measurement value. For example, the calculator is configured to set an average value, a minimum value, or a maximum value calculated based on each of the dynamic pressure signal and the static pressure signal obtained in a certain period from the start of measurement as the standard value of each of the dynamic pressure signal and the static pressure signal. Accordingly, since it is reliable that a user of the bed 110 is the subject 120, it is possible to reliably calculate the standard value corresponding to the subject 120. Alternatively, the calculator may set an average value, a minimum value, or a maximum value calculated based on each of the dynamic pressure signal and the static pressure signal obtained in a certain period from a timing of each predetermined time as the standard value of each of the dynamic pressure signal and the static pressure signal.

Accordingly, even when the user of the bed 110 changes from the subject 120 to another subject, a standard value corresponding to the other subject can be calculated.

The respective standard values are reference values for the dynamic pressure signal and the static pressure signal in the measurement of respiration and heartbeat. For each of the dynamic pressure signal and the static pressure signal, reliability of a measurement value is considered to be higher as a difference between the measurement value and the standard value is smaller and lower as the difference is larger. For example, the analyzer 340 is configured to compare a standard value with each of the dynamic pressure signal and the static pressure signal, and when a difference between the standard value and each of the dynamic pressure signal and the static pressure signal is equal to or greater than a predetermined measurement threshold, the analyzer 340 sets a corresponding one of the dynamic pressure signal and the static pressure signal as an object to be notified (alarm output) to the user. Accordingly, the user can recognize that the measurement value of the dynamic pressure signal and/or the static pressure signal deviates from the standard value. The predetermined measurement threshold is stored in, for example, the ROM 342 in advance.

The analyzer 340 further functions as a determiner, and is configured to determine a state of the subject 120 such as an in bed position, a body position (decubitus position), turning over, and out of bed and in bed, based on the dynamic pressure signal and/or the static pressure signal. For example, the determiner is configured to determine the in bed and out of bed of the subject 120, based on the static pressure signal. In a case where the static pressure signal is greater than a predetermined determination threshold, the determiner determines that the subject 120 is in bed. In a case where the static pressure signal is less than the determination threshold, the determiner determines that the subject 120 is out of bed. The determination threshold may be stored in the ROM 342 in advance. In addition, the determiner can more accurately detect the state of the subject 120 by analyzing a combination of the dynamic pressure signal and the static pressure signal. For example, the determiner may be configured to determine that the subject 120 is in bed in a case where the respiration and/or heartbeat of the subject 120 can be checked, based on the dynamic pressure signal in addition to the static pressure signal being larger than the predetermined determination threshold.

The determiner may be configured to calculate the determination threshold used to determine the state of the subject 120, based on the dynamic pressure signal and/or the static pressure signal of the subject 120. For example, the analyzer 340 may be configured to calculate the determination threshold for determining in bed and out of bed of the subject 120, based on the dynamic pressure signal and the static pressure signal of the subject 120.

Further, the determiner is configured to calculate respective determination thresholds, based on the dynamic pressure signal and the static pressure signal of the subject 120 obtained at the start of pressure measurement by the sensor 310 or at predetermined time intervals. For example, at a timing when the subject 120 gets in bed and respiration measurement is started, the determiner is configured to calculate a determination threshold when determining in bed and out of bed of the subject 120, based on the dynamic pressure signal and the static pressure signal of the subject 120. Accordingly, an appropriate determination threshold according to weight of the subject 120 can be applied to the state determination. Alternatively, the determiner is configured to calculate a determination threshold when determining in bed and out of bed of the subject 120, based on the dynamic pressure signal and the static pressure signal obtained in a certain period from a timing of each predetermined time. Accordingly, even when the user of the bed 110 changes from the subject 120 to another subject, a determination threshold corresponding to the other subject can be applied.

Further, the analyzer 340 is configured to estimate the weight of the subject 120, based on a change in the static pressure signal. The analyzer 340 is configured to estimate the weight of the subject 120, based on a look-up table or a relational expression representing a relationship between a magnitude of the static pressure signal and the weight of the subject 120. For example, a user who is a medical worker measures weight and a magnitude of the static pressure signal corresponding to the weight for each of a plurality of subjects in advance, and stores the measured weight and the magnitude of the static pressure signal in the ROM 342 as a look-up table or a relational expression.

Further, the analyzer 340 is configured to determine whether a weight equal to or greater than a load capacity is applied to the mat 210 based on the static pressure signal. A value of the static pressure signal that corresponds to the load capacity is stored in the ROM 342 in advance.

Further, the analyzer 340 is configured to determine whether an estimated value of the weight of the subject 120 and a tendency of the respiratory waveform and the heartbeat waveform rapidly change. For example, in a case where the estimated value of the weight of the subject 120 rapidly changes, the subject 120 may have been out of bed or replaced with another person having a different weight. In a case where the tendency of the respiratory waveform and the heartbeat waveform rapidly changes, conditions of the subject 120 may have been changed or the person has been replaced with another person having a different tendency of the respiratory waveform and the heartbeat waveform.

In the present embodiment, the analyzer 340 further functions as a detector, and is configured to detect an abnormality in the path from the airbag to the sensor 310, based on a change in the dynamic pressure and/or the static pressure. A specific method for detecting the abnormality will be described in detail later.

### Configuration of System 350

FIG. 4 is a schematic diagram illustrating a display screen of an operation display in FIG. 2. The system 350 can include a transmitter 351, an operation display 352, and a recorder 353.

The transmitter 351 is configured to transmit, to a terminal device 400, data such as an analysis result of respiration and heartbeat (respiratory rate and heart rate, respiratory waveform and heartbeat waveform, trend graph, and the like) by the analyzer 340, a state of the subject 120, an alarm, and an abnormality of a measurement path. The state of the subject 120 may include, for example, in bed and out of bed, a body position (decubitus position), a trend graph of a change in the body position, a position, estimated weight, and a health state of the subject 120. The user can check the data transmitted from the transmitter 351 in the terminal device 400 and store the data in a storage device. The terminal device 400 may be, for example, a personal computer, a smartphone, or a tablet terminal.

The transmitter 351 and the terminal device 400 are communicably connected to each other via a wireless and wired network. Examples of the network can include a local area network (LAN), a wide area network (WAN), and a USB. As a communication standard of the network, for example, Ethernet (registered trademark), Wi-Fi (registered trademark), Bluetooth (registered trademark), specific low-power radio for medical telemeter, or 5G may be used.

The operation display 352 can include, for example, a touch panel and various keys and switches, and receives instructions from the user, various settings, information on the patient (subject 120), and the like. The instructions by the user can include, for example, an instruction to start and end measurement, and the various settings can include, for example, settings related to a data output method or display method. The information on the patient can include sex, age, medical history, and the like of the patient.

The operation display 352 can include a display 360 and a speaker (not illustrated) disposed on one surface of a case of the physiological information measurement device 300, and is configured to output data such as an analysis result of respiration and heartbeat by the analyzer 340, a state of the subject 120, an alarm, and an abnormality of a measurement path. The output of the operation display 352 can include, for example, display of data on a display, audio output to a speaker, and print output to a printer. The operation display 352 may be connected to an external printer. FIG. 4 illustrates a case where a trend graph TG1 of the respiratory rate and a trend graph TG2 of the heart rate are displayed on the display 360. Details of data display on the display 360 will be described later.

The recorder 353 can include a large-capacity storage device such as an SSD, and is configured to record data.

### Physiological Information Measurement Method

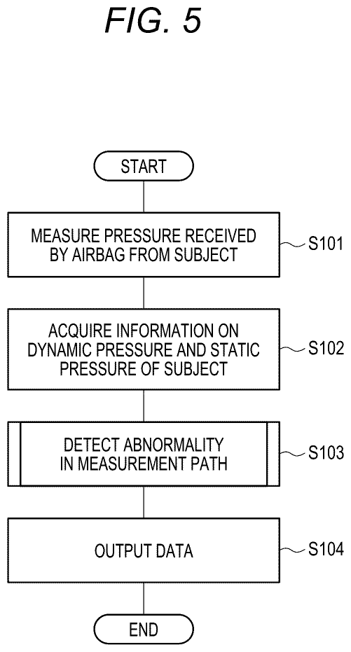

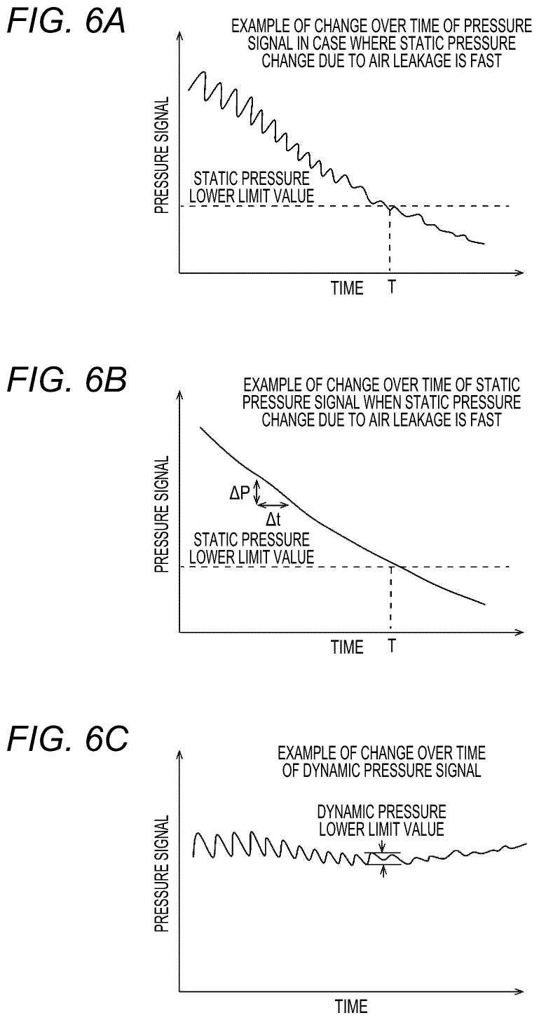

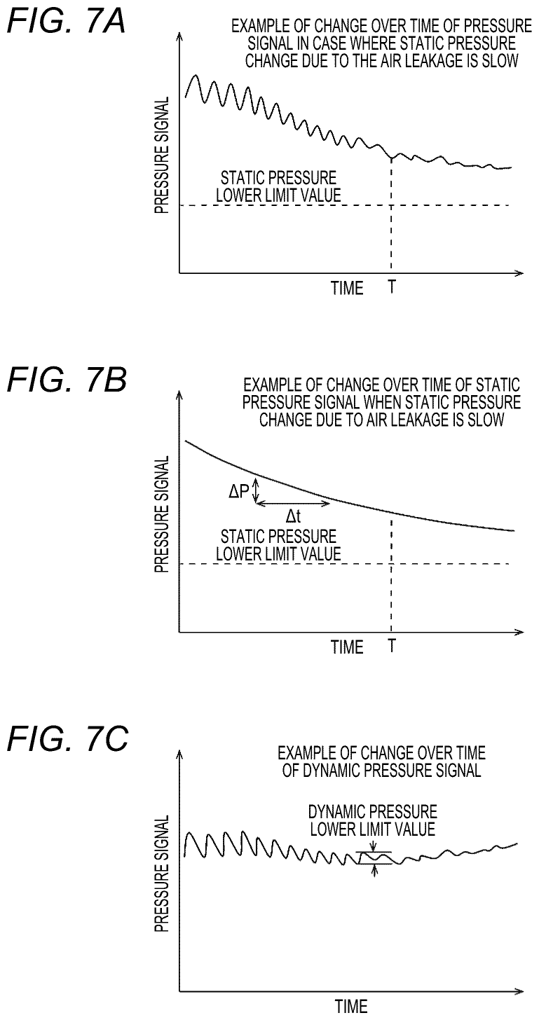

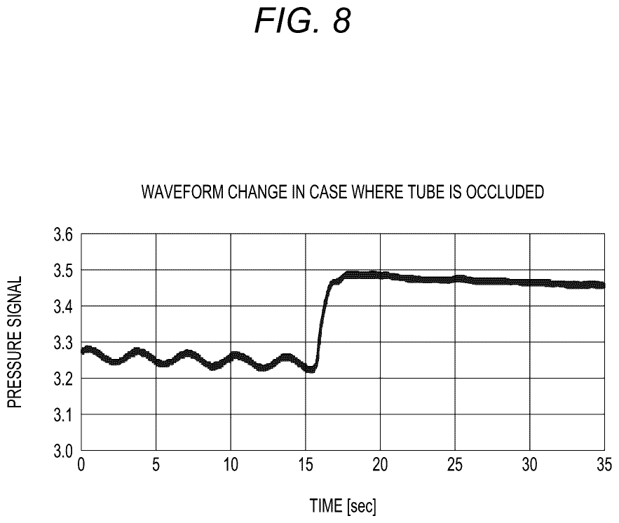

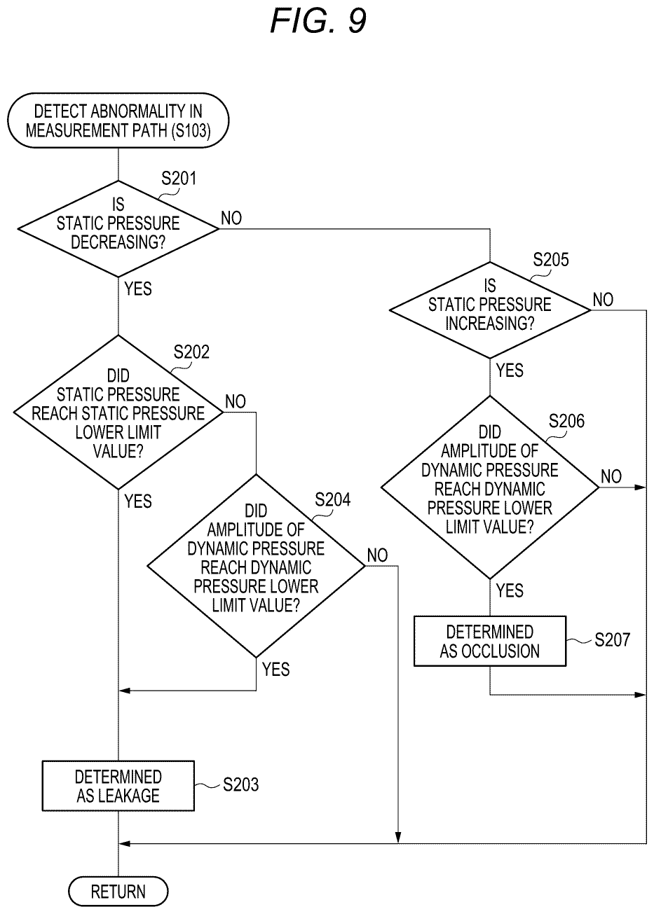

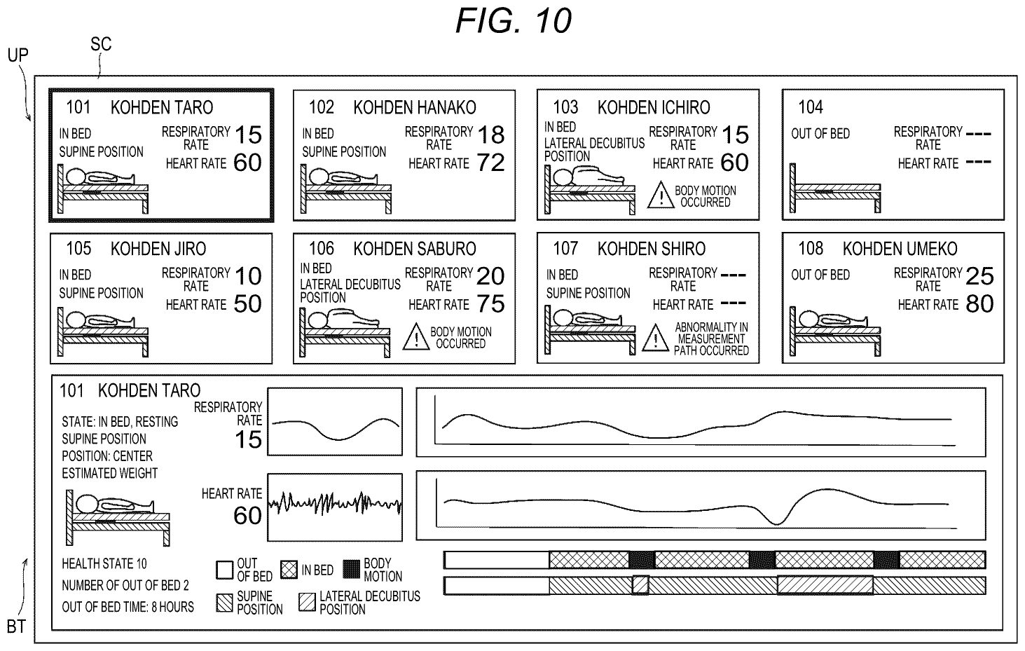

Hereinafter, an outline of a processing procedure of a physiological information measurement method by the physiological information measurement device 300 will be described with reference to FIGS. 5 to 10. FIG. 5 is a flowchart illustrating the outline of the processing procedure of the physiological information measurement method by the physiological information measurement device 300 according to the present embodiment. The processing of the flowchart in FIG. 5 is implemented by executing a physiological information measurement program by the CPU 341. FIGS. 6A to 6C are graphs respectively illustrating an example of a change over time of a pressure signal, an example of a change over time of a static pressure signal, and an example of a change over time of a dynamic pressure signal when a static pressure change due to air leakage is fast. FIGS. 7A to 7C are graphs respectively illustrating an example of a change over time of the pressure signal, an example of a change over time of the static pressure signal, and an example of a change over time of the dynamic pressure signal when the static pressure change due to air leakage is slow. FIG. 8 is a graph illustrating a waveform change when a tube is occluded. FIG. 9 is a subroutine flowchart illustrating processing details of step S103 in the flowchart in FIG. 5. The processing of the flowchart in FIG. 5 is implemented by executing a physiological information measurement program by the CPU 341. FIG. 10 is a schematic diagram illustrating data display on the display 360.

### Preparation of Measurement

First, before measurement of the respiratory rate of the subject 120, the user disposes the mat 210 in a position corresponding to the back (chest and abdomen) of the subject 120 between the lower surface of the mattress 100 and the upper surface of the bed board of the bed 110 or on the mattress 100. After the subject 120 gets on the bed 110, the user instructs the physiological information measurement device 300 to start measurement (for example, presses a measurement start button). Alternatively, the measurement is automatically started when power of the physiological information measurement device 300 is turned on.

### Measurement of Respiratory Rate

As in FIG. 5, when the measurement is started, the sensor 310 measures the pressure from the airbag, which is generated due to the body motion of the subject 120 (step S101). The first sensor 311 is used to measure the dynamic pressure of the subject 120, and the second sensor 312 is used to measure the static pressure of the subject 120.

Next, the signal processor 320 and the A/D converter 330 function as a processor, and obtain information on the dynamic pressure and the static pressure of the subject 120 (step S102). The processor processes the measurement result of the sensor 310 to obtain digital signals of the dynamic pressure and the static pressure (dynamic pressure signal and static pressure signal) as information on the dynamic pressure and the static pressure of the subject 120.

Next, the analyzer 340 detects an abnormality in the measurement path (path from the airbag to the sensor 310) (step S103). The analyzer 340 functions as a detector, and detects the abnormality in the measurement path, based on a change in the dynamic pressure and/or the static pressure. In the present embodiment, a case where air leakage (hereinafter also referred to as “leakage”) and occlusion are detected as the abnormality will be described below as an example.

### Details of Processing of Detection of Abnormality in Measurement Path (S103)

### <Leakage Detection Method>

When leakage occurs, air in the airbag is discharged into the atmosphere and thus the static pressure changes (decreases). A leakage detection method will be described in two cases including a case where the static pressure change due to leakage is fast and a case where the static pressure change due to leakage is slow. For example, as in FIG. 6A, in a case where the static pressure change due to leakage is fast, the pressure signal rapidly decreases (lowers) with elapse of time and reaches a static pressure lower limit value at time T. Here, the static pressure lower limit value is a value of the pressure signal corresponding to a lowest pressure measurable by the detector, and is stored in the ROM 342. On the other hand, as in FIG. 7A, in a case where the static pressure change due to leakage is slow, the pressure signal slowly decreases with the elapse of time, and does not reach the static pressure lower limit value at the time T. In the two cases, the detector detects leakage as follows.

(A) In the case where the static pressure change due to leakage is fast, as in FIG. 6B, the detector determines that leakage is detected when the static pressure signal rapidly decreases and falls below the static pressure lower limit value. Thus, leakage can be detected when the static pressure change due to leakage is fast. A speed at which the static pressure signal rapidly decreases may be, for example, a speed at which the static pressure signal decreases to 1 to 30%, typically 10% of an initial value in about seconds to tens of minutes.

(B) In the case where the static pressure change due to leakage is slow, as in FIGS. 7B and 7C, the detector determines that leakage is detected when the static pressure signal slowly decreases and decreases at a constant speed (that is, continuously) even before reaching the static pressure lower limit value, and an amplitude of the dynamic pressure signal decreases at a constant speed (that is, continuously) over a predetermined period and reaches a dynamic pressure lower limit value. Accordingly, leakage can be detected when the static pressure change due to leakage is slow. Here, the dynamic pressure lower limit value is a lower limit value set in advance for the amplitude of the dynamic pressure signal, and is stored in the ROM 342. The constant speed at which the static pressure signal decreases may be, for example, a speed at which the static pressure signal decreases to 30% to 70%, typically about 50% in hours. The constant speed at which the amplitude of the dynamic pressure signal decreases may be, for example, a speed at which the amplitude of the dynamic pressure decreases to 1% to 30%, typically about 10% in hours.

### <Occlusion Detection Method>

An occlusion detection method when occlusion occurs in any part of the measurement path will be described. For example, as in FIG. 8, in a case where the tube 220 is occluded, the pressure rapidly increases immediately after the occlusion. In the example in

FIG. 8, it is considered that the pressure signal rapidly increases immediately after 15 [sec] after the tube 220 is occluded. The value of the pressure signal in FIG. 8 is a value corresponding to a voltage value after being processed in the signal processor 320, and is used herein for a purpose of relatively representing a magnitude of the pressure signal.

(C) In a case where occlusion occurs in any part of the measurement path, the detector determines that occlusion is detected when the static pressure signal rapidly increases and the amplitude of the dynamic pressure signal decreases at a constant speed. Thus, occlusion in the measurement path can be detected. Here, the speed at which the static pressure signal increases accompanying the occlusion of the tube 220 is, for example, about seconds (typically, 1 to 2 [sec]).

In the present embodiment, the detector can detect an abnormality in the measurement path by performing the processing (A) to (C) independently or in an appropriate combination in parallel. As in FIG. 9, by combining the processing (A) to (C) to organize the determination for the static pressure signal and the dynamic pressure signal, it is possible to summarize the determination into a flowchart described as a series of processing procedures.

In the flowchart in FIG. 9, first, the detector determines whether the static pressure is decreasing (step S201). The detector determines that the static pressure is decreasing when a change rate of the static pressure signal relative to time is negative, and determines that the static pressure is increasing when the change rate is positive.

When the static pressure is decreasing (step S201: YES), the detector determines whether the static pressure reaches the static pressure lower limit value (step S202). When the static pressure reaches the static pressure lower limit value (step S202: YES), the detector determines that leakage occurs in any portion of the measurement path (step S203). On the other hand, when the static pressure does not reach the static pressure lower limit value (step S202: NO), the detector determines whether the amplitude of the dynamic pressure reaches the dynamic pressure lower limit value (step S204). When the amplitude of the dynamic pressure reaches the dynamic pressure lower limit value (step S204: YES), the detector determines that leakage occurs in any portion of the measurement path (step S203). On the other hand, when the amplitude of the dynamic pressure does not reach the dynamic pressure lower limit value (step S204: NO), the process is ended (returned).

On the other hand, when the static pressure is not decreasing (step S201: NO), the detector determines whether the static pressure is increasing (step S205), and ends the process (returns) when the static pressure is not increasing (step S205: NO). On the other hand, when the static pressure is increasing (step S205: YES), the detector determines whether the amplitude of the dynamic pressure reaches the dynamic pressure lower limit value (step S206), and determines that occlusion occurs in any part of the measurement path (step S207) when the amplitude of the dynamic pressure reaches the dynamic pressure lower limit value (step S206: YES). On the other hand, when the amplitude of the dynamic pressure does not reach the dynamic pressure lower limit value (step S206: NO), the process is ended (returned).

### Data Display

Returning to the flowchart in FIG. 5, the operation display 352 outputs data (step S104). For example, the operation display 352 causes the display 360 to display data such as an analysis result of respiration and heartbeat (respiratory rate and heart rate, waveform, trend graph, and the like) by the analyzer 340, a state of the subject 120, an alarm, and an abnormality in a respiration measurement path.

As in FIG. 10, the operation display 352 displays, for each patient, in an upper portion UP of a screen of the terminal device 400, of a screen SC of the display 360, or the like, information on a respiratory rate, a heart rate, in bed and out of bed, a body position (decubitus position), and an alarm of the patient (subject 120), together with a hospital room number of the patient. In the example in FIG. 10, an alarm indicating a body motion is displayed as information on an alarm for Mr. Kohden Ichiro in Room No. 103 and Mr. Kohden Saburo in Room No. 106, and an alarm indicating an abnormality in the measurement path is displayed as information on an alarm for Mr. Kohden Shiro in Room No. 107. The operation display 352 displays a body motion mark as the alarm of the body motion, when a motion of the subject 120 is rapid and a waveform is disordered, for example.

In the present embodiment, the analyzer 340 is configured to determine the in bed and out of bed of the subject 120, based on a change in the static pressure signal and/or the dynamic pressure signal, and the operation display 352 accurately displays the respiratory rate and the heart rate only when the subject 120 is in bed. This prevents the respiratory rate and the heart rate from being erroneously calculated and displayed even when the bed user is out of bed.

In a case where the analyzer 340 determines that a weight equal to or greater than the load capacity is applied to the mat 210, the operation display 352 notifies the user of the determination. Accordingly, it is possible to prevent the measurement of respiration and heartbeat from continuing in a state in which a weight equal to or greater than the load capacity is applied to the mat 210 and accuracy cannot be guaranteed.

In a case where a difference between the measurement value and the standard value is equal to or greater than the predetermined measurement threshold for each of the dynamic pressure signal and the static pressure signal, the operation display 352 notifies the user.

In addition, in a case where it is determined that the estimated value of the weight of the subject 120 and a tendency of the respiratory waveform and the heartbeat waveform rapidly change, the operation display 352 notifies the user of this determination. In a case where the estimated value of the weight of the subject 120 and the tendency of the respiratory waveform and the heartbeat waveform rapidly change, for example, conditions of the subject 120 may have changed or the subject 120 may have been replaced with another person, and thus the operation display 352 may be configured to notify the user of the change of the conditions of the subject 120 and the replacement of the user of the bed 110.

The operation display 352 is configured to display, for Mr. Kohden Taro in Room No. 101 in a bottom portion BT of the screen SC, in addition to the information in the upper portion UP, detailed information relating to information on in bed and out of bed, a position and estimated weight (specific numerical value is omitted in the figure) of Mr. Kohden Taro on the bed, graphs of respiration and heartbeat, information on health conditions, information on the decubitus position, and the like. From the data displayed on the screen SC, the user can immediately grasp that Mr. Kohden Taro is in a resting state and is in bed in a supine position at a center of the bed, and the respiratory rate is 15, the heart rate is 60, the health state is 10,the number of times of getting out of bed is 2, and out of bed time is 8 hours. Instead of the number of out of bed times and out of bed time, the number of in bed times and in bed time may be displayed. The information on health conditions is, for example, an index (score) evaluated on a scale of 1 to 10 relative to the health conditions of the subject 120, and is calculated based on changes in the respiratory rate and the heart rate and the respiratory waveform and the heartbeat waveform.

The graphs of respiration and heartbeat may be, for example, real-time waveforms of respiration and heartbeat or trend graphs of respiratory rate and heart rate. In the example in FIG. 10, for example, histories of in bed, out of bed, and the body motion in one day are displayed in band graph shapes as the information on in bed and out of bed, and a history of the body position, which is decubitus position (supine position or lateral decubitus position), in one day is displayed in a band graph shape as the information on the body position (decubitus position).

In the present embodiment, the state (in bed position, body position (decubitus position), out of bed and in bed) of the subject 120, the respiratory waveform and the heartbeat waveform, and the respiratory rate and the heart rate are displayed together.

Reliability of calculated numerical values may also be displayed. Accordingly, the user (medical worker) can accurately determine the condition change of the subject 120. In addition, a possibility of a bedsore may be determined from the body position (decubitus position) and the number of body position changes of the subject 120. In addition, a quality of sleep may be evaluated from a change in the body position (decubitus position) of the subject 120 during sleep and a change in the respiratory waveform and the heartbeat waveform.

In this manner, in the processing of the flowchart in FIG. 5, the sensor 310 measures the pressure received by the airbag containing air from the subject 120, and the signal processor 320 processes the measurement result to obtain information on the dynamic pressure and the static pressure of the subject 120. Then, the analyzer 340 detects an abnormality in the measurement path based on a change in the dynamic pressure and/or the static pressure. The processing of the flowchart in FIG. 5 (processing of steps S101 to S104) is repeatedly executed during a measurement period.

### First Modification

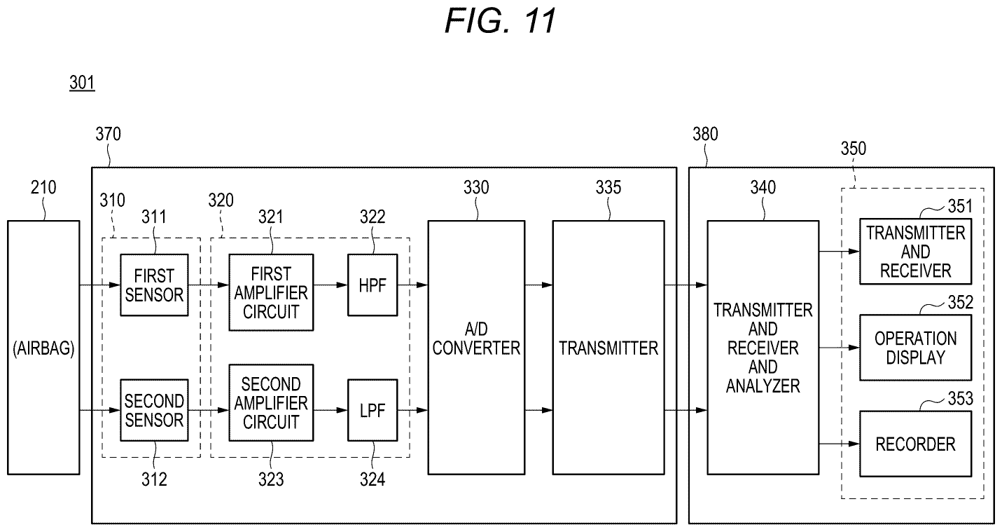

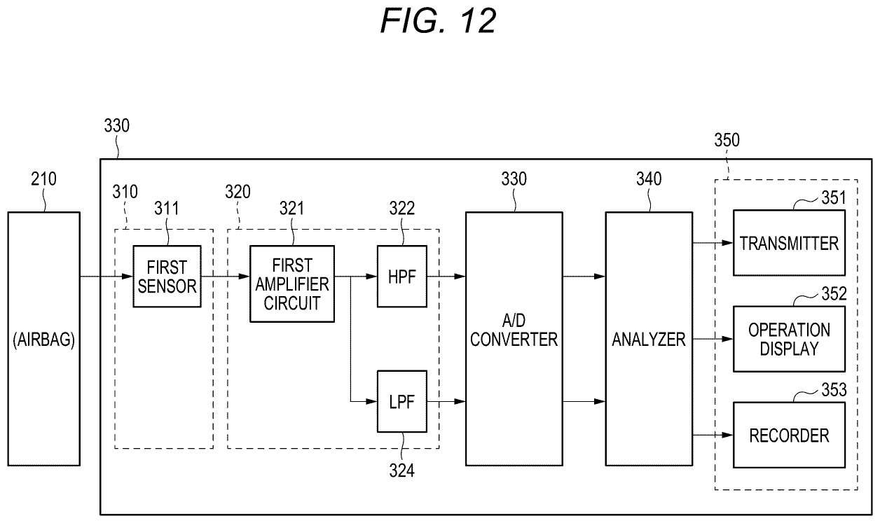

FIG. 11 is a block diagram illustrating a schematic configuration of the physiological information measurement device 300 in FIG. 1 according to a first modification.

The first modification describes a case where a physiological information measurement device 301 include two portions including a first body 370 that includes the sensor 310, the signal processor 320, the A/D converter 330, and a transmitter 335, and a second body 380 that includes the transmitter and receiver and analyzer 340 and the system 350. The first body 370 and the second body 380 may be disposed in different places in a hospital, for example, and may communicate with each other. For example, the first body 370 is disposed on the bed 110 in a hospital room of a patient, and the second body 380 may be carried by a user. The second body 380 may be a computer such as a server. The computer functions as the transmitter and receiver and analyzer 340 and the system 350.

Configurations of the sensor 310, the signal processor 320, and the A/D converter 330 are the same as corresponding configurations in the physiological information measurement device 300, and thus detailed descriptions thereof will be omitted. The transmitter 335 is configured to transmit, to the second body 380, a static pressure signal and a dynamic pressure signal converted by the A/D converter 330. The transmitter and receiver and analyzer 340 of the second body 380 functions as a receiver or an obtainer, and is configured to receive the static pressure signal and the dynamic pressure signal transmitted by the transmitter 335. The transmitter and receiver and analyzer 340 is configured to calculate a respiratory rate, a heart rate, and the like of the subject 120, based on the dynamic pressure signal. The transmitter and receiver and analyzer 340 further functions as a detector, and is configured to detect an abnormality in a measurement path, based on a change in the dynamic pressure signal and/or the static pressure signal. Details of a method for calculating the respiratory rate, the heart rate, and the like, and a method for detecting an abnormality are the same as the above-described methods, and thus detailed descriptions thereof will be omitted.

According to the present modification, the user can check data of the subject 120 in a place different from the bed 110 in the hospital room in which the first body 370 is disposed.

### Second Modification

FIG. 12 is a block diagram illustrating a schematic configuration of the physiological information measurement device 300 in FIG. 1 according to a second modification.

The second modification describes a case where the first sensor 311 also serves as the second sensor 312. In this case, the first sensor 311 is also referred to as a third sensor. In the present modification, the sensor 310 can include the first sensor 311 and does not include the second sensor 312. The signal processor 320 can include the first amplifier circuit 321, the HPF 322, and the LPF 324, and does not include the second amplifier circuit 323. An output of the first amplifier circuit 321 is connected to inputs of the HPF 322 and the LPF 324, and the LPF 324 is configured to extract an AC (or DC) component equal to or lower than a second cutoff frequency fc2 from an electric signal amplified by the first amplifier circuit 321 and output the AC component to the A/D converter 330. That is, in the present modification, a dynamic pressure component measured by the first sensor 311 is extracted by the HPF 322, and a static pressure component measured by the first sensor 311 is extracted by the LPF 324.

According to the present modification, the second sensor 312 and the second amplifier circuit 323 can be omitted, and the number of components of the physiological information measurement device is reduced, and thus costs in manufacturing the physiological information measurement device can be reduced.

Although the case where the sensor 310 can include only the first sensor 311 (third sensor) also serving as the second sensor 312 is described, the presently disclosed subject matter is not limited to such a case, and the sensor 310 may include the first to third sensors.

### Third Modification

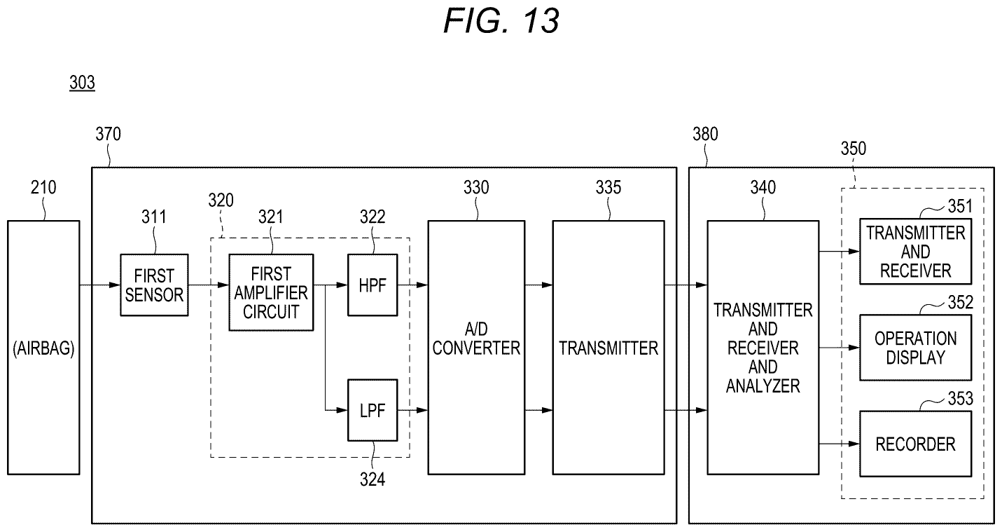

FIG. 13 is a block diagram illustrating a schematic configuration of the physiological information measurement device 300 in FIG. 1 according to a third modification.

The third modification is a modification combining the first modification and the second modification. That is, a physiological information measurement device 303 can include two portions including the first body 370 and the second body 380. The sensor 310 can include the first sensor 311 and does not include the second sensor 312. The signal processor 320 can include the first amplifier circuit 321, the HPF 322, and the LPF 324, and does not include the second amplifier circuit 323.

Physiological information measurement devices 300 to 303 according to the present embodiments described above have the following effects.

An abnormality in the measurement path from the airbag to the sensor is detected based on a change in the dynamic pressure and/or the static pressure of the subject 120. Accordingly, an abnormality in the measurement path can be detected.

The analyzer 340 can distinguish between a case where the subject 120 is out of bed and a case where an abnormality occurs in the measurement path (for example, a case where the tube 220 is detached) by performing analysis by combining the dynamic pressure signal and the static pressure signal.

The analyzer 340 performs analysis by combining the dynamic pressure signal and the static pressure signal, so that it is possible to accurately detect the state of the subject 120, for example, rising, turning over, and out of bed of the subject 120.

Since the measurement performance is prevented from varying depending on the in bed position and the body position (decubitus position) of the subject 120, it is possible to determine whether changes in the respiratory waveform and the heartbeat waveform and the respiratory rate and the heart rate are caused by a change in conditions or another factor (for example, a change in the body position and a lying position).

The physiological information measurement devices 300 to 303, the physiological information measurement method, and the physiological information measurement program according to the embodiment of the presently disclosed subject matter are described above. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

For example, in the examples described above, the signal processor 320 is implemented by an analog circuit, and is disposed in front of the A/D converter 330. Alternatively, the presently disclosed subject matter is not limited thereto, and the signal processor may be implemented by a digital circuit and disposed behind the A/D converter 330.

In the examples described above, the physiological information measurement program is executed by the analyzer 240 (computer) in the physiological information measurement devices 300 to 303, and the respiratory rate, the heart rate, and the like of the subject 120 are calculated and an abnormality in the measurement path is detected. Alternatively, the presently disclosed subject matter is not limited to this case. For example, the calculation processing of the respiratory rate, the heart rate, and the like and the abnormality detection in the measurement path may be performed by a server or a general-purpose computer (personal computer, smartphone, tablet terminal, or the like) outside the physiological information measurement devices 300 to 303.

A part or all of the functions implemented by the physiological information measurement program in the embodiment described above may be implemented by hardware such as an electric circuit.

For example, a configuration below also constitutes a part of the present disclosure:

A non-transitory computer readable storage medium storing a physiological information measurement program including instructions which, when executed by the computer, cause the computer to execute processing included in a physiological information measurement method,
wherein the physiological information measurement method includes:
   measuring, by a sensor, pressure received by an airbag containing air from a subject;
   processing a measurement result in the measuring and obtaining information on a dynamic pressure and a static pressure of the subject; and
   detecting an abnormality in a path from the airbag to the sensor, based on a change in the dynamic pressure and/or the static pressure.

What is claimed is:

1. A physiological information measurement device comprising:
a sensor connected to an airbag containing air, the sensor being configured to measure pressure received by the airbag from a subject;
a processor configured to process a measurement result of the sensor to obtain information on a dynamic pressure and a static pressure of the subject; and
a detector configured to detect an abnormality in a path from the airbag to the sensor, based on a change in the dynamic pressure and/or the static pressure.

2. The physiological information measurement device according to claim 1,
wherein, in a case where the static pressure of the subject decreases and falls below a predetermined static pressure lower limit value, or in a case where the static pressure of the subject continues decreasing for a predetermined period and an amplitude of the dynamic pressure reaches a predetermined dynamic pressure lower limit value, the detector determines, as the abnormality, that air leakage in the path from the airbag to the sensor occurs.

3. The physiological information measurement device according to claim 1,
wherein, in a case where the static pressure of the subject increases and the amplitude of the dynamic pressure of the subject reaches a predetermined dynamic pressure lower limit value, the detector determines, as the abnormality, that an occlusion in the path from the airbag to the sensor occurs.

4. The physiological information measurement device according to claim 1, further comprising:
a determiner configured to determine a state of the subject, based on the information on the dynamic pressure and/or the static pressure of the subject.

5. The physiological information measurement device according to claim 1, further comprising:
a calculator configured to calculate a standard value for each of the information on the dynamic pressure and the static pressure of the subject.

6. The physiological information measurement device according to claim 5,
wherein the calculator is configured to calculate the respective standard values, based on the information on the dynamic pressure and the static pressure of the subject that are obtained at a start of measurement of pressure by the sensor or at predetermined time intervals.

7. The physiological information measurement device according to claim 5,
wherein the calculator is configured to compare the information on the dynamic pressure and the static pressure of the subject with the respective standard values, and
in a case where there is a state where a difference between the information on the dynamic pressure and the static pressure of the subject, and the respective standard values is equal to or greater than a predetermined threshold, the state is notified to a user.

8. The physiological information measurement device according to claim 1,
wherein the sensor includes:
a first sensor configured to measure the dynamic pressure of the subject; and
a second sensor configured to measure the static pressure of the subject.

9. The physiological information measurement device according to claim 1,
wherein the sensor includes a third sensor configured to measure the dynamic pressure and the static pressure of the subject.

10. The physiological information measurement device according to claim 4,
wherein the determiner is configured to calculate a determination threshold used for determining the state of the subject, based on the information on the dynamic pressure and/or the static pressure of the subject.

11. The physiological information measurement device according to claim 10,
wherein the determiner is configured to calculate the respective determination thresholds, based on the information on the dynamic pressure and the static pressure of the subject that are obtained at a start of measurement of pressure by the sensor or at predetermined time intervals.

12. A physiological information measurement device comprising:
a first body; and
a second body,
wherein the first body includes:
a sensor connected to an airbag containing air, and the sensor being configured to measure pressure received by the airbag from a subject;
a processor configured to process a measurement result of the sensor to obtain information on a dynamic pressure and a static pressure of the subject; and
a transmitter configured to transmit, to the second body, the information on the dynamic pressure and the static pressure of the subject, and
the second body includes:
a receiver configured to receive, from the first body, the information on the dynamic pressure and the static pressure of the subject; and
a detector configured to detect an abnormality in a path from the airbag to the sensor, based on a change in the dynamic pressure and/or the static pressure.

13. The physiological information measurement device according to claim 12,
wherein the second body is a computer configured to function as the receiver and the detector.

14. A physiological information measurement device for measuring physiological information on a subject, based on pressure received by an airbag containing air from the subject, the physiological information measurement device comprising:
an obtainer configured to obtain information on a dynamic pressure and a static pressure of the subject; and
a detector configured to detect an abnormality in a path from the airbag to a sensor, based on a change in the dynamic pressure and/or the static pressure, the sensor being connected to the airbag and configured to measure the pressure received by the airbag from the subject.

15. A physiological information measurement method comprising:
measuring, by a sensor, pressure received by an airbag containing air from a subject;
processing a measurement result in the measuring and obtaining information on a dynamic pressure and a static pressure of the subject; and
detecting an abnormality in a path from the airbag to the sensor, based on a change in the dynamic pressure and/or the static pressure.

16. The physiological information measurement method according to claim 15,
wherein in the detecting, in a case where the static pressure of the subject decreases and falls below a predetermined lower limit value, or in a case where the static pressure of the subject continuously decreases for a predetermined period and an amplitude of the dynamic pressure continuously decreases for a predetermined period, it is determined that air leakage in a path from the airbag to the sensor occurs as the abnormality.

17. The physiological information measurement method according to claim 15,
wherein in the detecting, in a case where the static pressure of the subject increases and an amplitude of the dynamic pressure of the subject decreases, it is determined that occlusion in a path from the airbag to the sensor occurs as the abnormality.
